(19) United States
(12) Patent Application Publication
(10) Pub. No.: US 2021/0401343 A1
(43) Pub. Date: Dec. 30, 2021

(54) WEARABLE APPARATUS AND DRY ELECTRODE FOR ACQUIRING ELECTROPHYSIOLOGICAL SIGNALS AS WELL AS METHOD FOR PRODUCING THE SAME

(71) Applicant: Fraunhofer-Gesellschaft zur Förderung der angewandten Forschung e.V., Munich (DE)

(72) Inventors: Ruslan RYBALKO, Erlangen (DE); Christian HOFMANN, Erlangen (DE)

(21) Appl. No.: 17/362,516

(22) Filed: Jun. 29, 2021

(30) Foreign Application Priority Data
Jun. 30, 2020 (DE) 102020208157.3

(51) Int. Cl. A61B 5/256; A61B 5/27; A61B 5/28; A61B 5/268; A61B 5/265

(52) U.S. Cl. CPC A61B 5/256; A61B 5/27; A61B 2562/125; A61B 5/268; A61B 5/265; A61B 5/28

(57) ABSTRACT

Embodiments provide a wearable apparatus for acquiring electrophysiological signals of a living being. The apparatus includes a textile carrier, at least two dry electrodes attached to an inside of the textile carrier and at least two adjustable straps attached to the textile carrier that allow to adjust the wearable apparatus to a body of the living being and a contact pressure of the at least two dry electrodes to a skin of the living being.

### CROSS-REFERENCE TO RELATED APPLICATIONS

This application claims priority from German Patent Application No. 102020208157.3, which was filed on Jun. 30, 2020, and is incorporated herein in its entirety by reference.

Embodiments of the present invention relate to a wearable apparatus for acquiring electrophysiological signals. Further embodiments relate to a dry electrode for electrophysiological signals. Further embodiments relate to a method for producing a wearable apparatus for acquiring electrophysiological signals.

### BACKGROUND OF THE INVENTION

For acquiring electrophysiological signals of living beings, such as at the human body, good contacting of the primary measurement value sensors (e.g. electrodes) is needed for obtaining good signal quality. Above that, this adaptation also has to show sufficient robustness since interferences occur due to electrodes not being applied evenly during signal acquisition in everyday life and while moving (motion artefacts).

If looking more closely at the signal processing chain, different functional blocks are linked for signal acquisition from the body up to evaluation electronics:
the electrode area has to be conductive and has to have a resistance as small as possible to capture the physiological signals with good quality,
the conductive portion is to transmit the captured measurement values possibly without interference up to the contacting location with the evaluation electronics and
all used components and materials are to have high wearing comfort and biocompatibility.

In the sense of a good adaptation, the measurement value sensors are to ensure good signal acquisition for different body sizes, different body proportions and for different sexes. Therefore, textile carrier systems including integrated measurement value sensors (e.g. shirt or vest or strap) have to provide for these anatomic requirements.

When acquiring electrophysiological signals at the body, typically, silver-silver chloride adhesive electrodes (Ag/AgCl) are used in everyday clinical practice. These types of electrodes have an adhesive edge (similar to a Band-Aid) and a conductive core that is additionally provided with a contact gel so that low skin-electrode resistance can be obtained.

However, these electrodes have the following disadvantages:
When applying the electrodes, the gel feels unpleasant for the persons to be measured.
With longer measurement duration, the adhesive edge loses its adhesive effect due to natural transpiration, whereby the electrodes may fall off and no evaluable data can be captured anymore due to signal loss.
For prolonged application (e.g. 24-hour electrocardiogram) or measurement up to a week), skin irritations may occur.

For some years, research has been made with regard to different materials for dry electrodes that prevent the usage of contact gel due to high conductivity. In most cases, these dry electrodes are incorporated in textile carrier systems (pulse-chest strap, sensor shirt, vest, strap system). Sensor systems for a single-channel ECG are easy to implement. Here, two electrodes are integrated, for example, in a chest strap that is applied evenly to the body at most times and provides stable signals. Much higher requirements are placed on sensor systems for recording multichannel ECG, since here several electrodes (e. g. four electrodes for a 3-channel ECG or seven electrodes for a 9-channel ECG or ten electrodes for a 12-channel ECG) are to be integrated into the textile at different application locations and sufficiently good adaptation has to be ensured at all times.

Here, the textile carriers also have to provide for the already above-mentioned anatomic requirements of the wearers (e.g. sex, size, proportions, limitations).

Disadvantages of previous textile-integrated sensor systems:
No sufficiently good signals due to poor electrode materials.
No sufficiently good signals due to low adaptation (e.g. contact pressure too low).
No sufficiently good signals due to motion artefacts.
Used construction and materials not washable, no rendering possible.
No long-term stability of signal quality (conductance of the electrode material decreases, e.g. during washing).
Specific design for women and men needed.
Many different clothing sizes needed.
Sensor system too noticeable and hence not accepted.
Sensor system uncomfortable to wear in everyday life.
Sensor system uncomfortable to wear during sleep analysis.
Sensor system not adjustable.
Sensor system cannot be put on by the person alone.
Sensor system results in strong limitations in the choice of clothes.

An important point of monitoring in everyday life is the sufficient acceptance and good applicability by the user. The textile carrier systems have to be easy to put on and to be adaptable by the user in a fast and uncomplicated manner at any time in everyday life.

### SUMMARY

According to an embodiment, a wearable apparatus for acquiring electrophysiological signals of a living being may have: a textile carrier, at least two dry electrodes attached to an inside of the textile carrier and at least two adjustable straps attached to the textile carrier, which allow adjusting the wearable apparatus to the body of the living being and a contact pressure of the at least two dry electrodes on a skin of the living being.

According to another embodiment, a dry electrode for acquiring electrophysiological signals of a living being may have: a layer of conductive fabric and a layer of electrically conductive polymer covering the layer of conductive fabric.

According to another embodiment, a method for producing a wearable apparatus for acquiring electrophysiological signals of a living being may have the steps of: providing a textile carrier, forming at least two dry electrodes on an inside of the textile carrier by means of a transfer printing method, providing at least two adjustable straps and attaching the at least two adjustable straps to the textile carrier.

According to another embodiment, a method for acquiring a multi-channel electrocardiogram of a living being by means of an inventive wearable apparatus may have the steps of: applying a reference signal to the living being with a dry electrode of the at least four dry electrodes of the wearable apparatus, acquiring at least three electrophysiological signals from the living being using at least three other dry electrodes of the at least four dry electrodes of the wearable apparatus, processing the acquired at least three electrophysiological signals to obtain the multi-channel electrocardiogram of the living being.

Embodiments provide a wearable apparatus for acquiring electrophysiological signals of a living being [e.g. animal, human being [e.g. human patient]]. The apparatus includes a textile carrier, at least two dry electrodes [e. g. two, three or four dry electrodes] attached to an inside of the textile carrier and at least two adjustable straps attached to the textile carrier, which allow the adjustment of the wearable apparatus to a body of the living being and a contact pressure of the at least two dry electrodes on the skin of the living being.

In embodiments, the at least two dry electrodes can be at least four dry electrodes for acquiring a multi-channel electrocardiogram of the living being.

In embodiments, the textile carrier can be made of an [e.g. single] piece of material.

In embodiments, the textile carrier can form the shape of a strap system together with the at least two straps.

In embodiments, the textile carrier can comprise a central area, two upper areas, each extending away from the central area, and two lateral areas, each extending away from the central area.

In embodiments, when the apparatus is worn by the living being, the central area of the textile carrier can extend across a back area of the living being, the two upper areas of the textile carrier can extend, starting from the back area, across the respective shoulder areas up to the upper chest areas of the living being and the two lateral areas of the textile carrier can extend, starting from the back area, across respective axillary lines up to the respective upper stomach areas or lower chest areas.

In embodiments, the two upper areas of the textile carrier can be connected to the two lateral areas of the textile carrier via two of the at least two straps.

In embodiments, the two lateral areas of the textile carrier can be connected to each other via a connecting element [e.g. Velcro fastener, buckle, clamping strap or push button].

In embodiments, the two lateral areas of the textile carrier can be connected to each other via a third strap of the at least two straps.

In embodiments, the at least two dry electrodes can be attached to the textile carrier on at least two areas of the two upper areas and the two lateral areas of the textile carrier.

In embodiments, the at least two dry electrodes can be at least four dry electrodes, wherein two dry electrodes of the at least four dry electrodes are attached to an inside of the two upper areas of the textile carrier, wherein two other dry electrodes of the at least four dry electrodes are attached to an inside of the two lateral areas of the textile carrier.

In embodiments, when the apparatus is worn by the living being, the two dry electrodes can contact clavicle areas [e.g., a left clavicle area and a right clavicle area] or upper areas above a chest area of the living being.

In embodiments, when the apparatus is worn by the living being, the two other dry electrodes can contact abdominal areas [e.g., within an abdominal quadrant] or lower regions below a chest area of the living being.

In embodiments, the textile carrier and/or the at least two straps can be elastic.

In embodiments, the at least two dry electrodes can each comprise a layer of conductive fabric [e.g. silvered fabric] and a layer of electrically conductive polymer [e. g. silicon] covering the layer of conductive fabric.

In embodiments, the layer of electrically conductive polymer can be thinner than 1 mm.

In embodiments, for example [e. g. different] layers of the at least two dry electrodes can be formed by means of a combination of a screen printing method [e. g. for providing the individual functional layers] and a transfer printing method [e. g. for packaging=welding together and integration in the textile carrier].

In embodiments, the at least two dry electrodes can be connected, via insulated lines, to a terminal [e.g. for providing the electrophysiological signals acquired with the at least two dry electrodes] attached to an outside of the textile carrier.

In embodiments, the insulated lines can each comprise a layer of conductive fabric and at least one layer of insulating material [e.g. only cover layer or also base and cover layer] covering the layer of conductive fabric.

In embodiments, layers of the insulated lines can be formed by means of a combination of a screen printing method and a transfer printing method.

In embodiments, the insulated lines can be connected each to a connecting element [e.g. push button, banana jack] guided to the outside of the terminal.

In embodiments, the terminal can comprise at least one layer of insulating material [e.g. only cover layer or also base and cover layer], wherein a layer of the at least one layer of insulating material is opened in areas of the connecting elements, such that the connecting elements are exposed.

Further embodiments provide a dry electrode for acquiring electrophysiological signals of a living being. The dry electrode includes a layer of conductive fabric [e.g. silvered fabric] and a layer of electrically conductive polymer [e.g. silicon] covering the layer of conductive fabric.

In embodiments, the conductive fabric can be a silvered fabric.

In embodiments, the layer of electrically conductive polymer [e.g. silicon] can have a thickness of less than 1 mm.

In embodiments, the dry electrode can further comprise a thermoplastic polyurethane film wherein the layer of conductive fabric is arranged on the thermoplastic polyurethane film.

In embodiments, the dry electrode can be embedded in a transfer printing film layer system of at least two transfer printing films, wherein the transfer printing film layer system is partly opened in an area adjacent to the layer of electrically conductive polymer [e. g. silicon], such that the layer of electrically conductive polymer is partly exposed.

In embodiments, the dry electrode can be attached to a textile carrier by means of a transfer printing method.

Further embodiments provide a method for producing a wearable apparatus for acquiring electrophysiological signals of a living being. The method includes a step of providing a textile carrier. Further, the method includes a step of forming at least two dry electrodes on an inside of the textile carrier by means of a transfer printing method. Further, the method includes a step of providing at least two adjustable straps and attaching the at least two adjustable straps to the textile carrier.

In embodiments, forming the at least two dry electrodes can comprise a step of providing a layer of electrically conductive fabric [e. g. silvered fabric] and a step of providing a layer of electrically conductive polymer [e. g. silicon] on the layer of electrically conductive fabric, such that the layer of electrically conductive polymer at least partly covers the layer of electrically conductive fabric.

In embodiments, forming the at least two dry electrodes can further comprise a step of providing a thermoplastic polyurethane film, wherein the layer of a conductive fabric is arranged on the thermoplastic polyurethane film.

In embodiments, forming the at least two dry electrodes can further comprise a step of providing a first transfer printing film and a second transfer printing film, wherein the layer of electrically conductive polymer and the layer of electrically conductive fabric are arranged between the first transfer printing film and the second transfer printing film, wherein the first transfer printing film is arranged on the layer of electrically conductive polymer, wherein the first transfer printing film is partly opened in an area adjacent to the layer of electrically conductive polymer, such that the layer of electrically conductive polymer is partly exposed.

In embodiments, the layer of electrically conductive polymer and the layer of electrically conductive fabric can be embedded between the first transfer printing film and the second transfer printing film by means of the transfer printing method.

In embodiments, when forming the at least two dry electrodes, at least two insulated lines can be formed.

In embodiments, the method can further comprise a step of forming a terminal [e.g., for providing the electrophysiological signals acquired with the at least two dry electrodes] on the outside of the textile carrier, wherein the terminal is connected to the at least two insulated lines.

In embodiments, when forming the terminal, connecting elements [e.g., push buttons or banana jacks] can be formed, which are each connected to one of the two insulated lines.

In embodiments, when forming the terminal, a further transfer printing film can be provided, wherein the further transfer printing film is opened in areas of the connecting elements guided to the outside, such that the connecting elements are exposed, wherein the further transfer printing film is attached to the outside of the textile carrier by means of a transfer printing method to form the terminal.

Further embodiments provide a method for acquiring a multi-channel electrocardiogram of a living being with a wearable apparatus having at least four dry electrodes according to one of the embodiments described herein. The method comprises a step of coupling a reference signal to the living being with a dry electrode of the at least four dry electrodes of the wearable apparatus. The method further comprises a step of acquiring at least three electrophysiological signals from the living being using at least three other dry electrodes of the at least four dry electrodes of the wearable apparatus to obtain the multi-channel electrocardiogram [e.g., by processing the acquired at least three electrophysiological signals].

Embodiments described herein provide a textile carrier by which both the above-described objectives regarding stable comfortable signal acquisition of physiological parameters can be implemented in everyday life and mobile usage, but can be realized without the disadvantages of conventional technologies.

A first aspect relates to a textile carrier with an adjustable strap system that can be worn similar to a vest or can be put on like a backpack. In embodiments, the straps can be adapted to the physical size and physical proportions of the user anytime, e.g., via a system of Velcro fasteners on both shoulders as well as on the stomach. Adapting the strap system mainly influences the adaptation of the primary measurement value sensors (ECG electrodes), such that sufficiently good skin electrode contact and hence high signal quality is ensured at all times. Adjustment does not have to take place prior to applying the textile carrier (such as the adjustment buckle in a pulse chest strap for initially changing the width), but can also be adapted to the activities and needs of the wearer during data acquisition. Due to the cutting pattern (no fabric sections at the front of the chest), the carrier textile is implicitly suitable both for men and women. Both the adjustability to the current situation and activity as well as the concept as sex-independent textile, support the aim of obtaining continuous good adaptation and hence, high signal quality during signal acquisition in everyday life (also across different phases during the day).

A second aspect supporting the aim of good signal quality relates to skillful selection and combination of materials that are best suited for signal acquisition, signal transmission and interference suppression. The transmission path from the primary measurement value sensors to the contacting location for the evaluation electronics is divided into the following functional blocks:
Electrode material in direct contact with the skin surface and acquiring the physiological parameters. The electrodes are to have high conductance, distinctive adhesive effect on the skin surface (no slipping off during movements) and biocompatibility.
The measurement line is to transmit the captured signals without loss and with low interference. This means that the used material and the built structure are to have a high conductance, significant elasticity and low change of resistance during mechanical influence.
The insulating layer protects the acquired signal in the measurement line from interference couplings.

It applies for all components that the needed characteristics may not be changed by chemical and mechanical influences during washing.

A third aspect for improving the quality of acquired signal relates to the way of integrating the selected materials into the textile carrier system. In embodiments, electrode material, measurement line and insulation layer are introduced in a multi-layered structure setup:
The lowest layer directly on the carrier textile is, on the one hand, a lower insulation level and, at the same time, the carrier surface for the entire system of measurement value sensor and measurement line (lower cladding).
The second layer is a conductive material (core).
The third layer is an electrically conductive and skin-friendly silicon placed only locally at the electrode area. For protecting their underlying conductive material from being washed out and, at the same time, for forming good signal transmission from the skin to silicon to measurement line (electrode).
The fourth layer is the upper insulating layer covering all areas of the still open measurement line (upper cladding) apart from the electrode area (already covered by the third layer).

The structure made up of the two cladding areas fulfills the following three objects in the textile carrier system:
Electric insulation and protection of the acquired signal and hence, prevention of interference signals and short circuits.
Mechanical stabilization of the measurement line and reduction of motion artifacts.
Protecting the conductive material from being washed out and hence maintaining the physical material characteristics.

For extended elasticity of the measurement lines during movements of the user, the same can be implemented in a meandering structure. During stretching and compressing, lower mechanical stress and hence low resistance modulation takes place.

Embodiments provide a textile carrier combining the above-described features in the following way:
Combination of suitable materials for a sensor system with dry electrodes having high wearing comfort and improved (e.g., best possible) signal quality.
Concept of an adjustable textile carrier for flexible and improve (e.g., good) adaptation.
Integration of the functional components for improved (e.g., highest) interference stability and low artifact susceptibility.
Additionally, the developed carrier system comprises simple handling, flexible adaptation, inconspicuousness and high wearing comfort, such that high user acceptance and hence both application-related improvements as well as commercial success can be reckoned with.

In embodiments, the used materials generate a signal transmission chain by which improved (e.g., best possible) signal qualities can be obtained.

In embodiments, the concept of the carrier textile allows an individual adaptability which is also flexible in time, such that sufficiently good adaptation and hence good signal acquisition is ensured also for different persons and activities.

In embodiments, the way of integration generates an improved (e.g., good) interference stability and artifact stability for the measurement line, long-term stability across longer usage duration during monitoring in everyday life (in particular with respect to washing cycles).

Embodiments of the present invention are applied in medical monitoring of patients with cardiovascular risk constellation, medical monitoring of patients during cardiological rehabilitation and in telemedical cardio monitoring.

### BRIEF DESCRIPTION OF THE DRAWINGS

Embodiments of the present invention will be detailed subsequently referring to the appended drawings, in which:

FIG. 1a is a schematic front view of an apparatus for acquiring electrophysiological signals of a living being according to an embodiment of the present invention,

FIG. 1b is a schematic rear view of the apparatus for acquiring electrophysiological signals of a living being according to an embodiment of the present invention,

FIG. 2a is an illustration of a front view of the apparatus for acquiring electrophysiological signals of a living being when the same is exemplarily worn by a doll as a representative for a living being,

FIG. 2b is an illustration of a rear view of the apparatus for acquiring electrophysiological signals of a living being when the same is worn exemplarily by a doll as a representative for a living being,

FIG. 3 is a schematic view of a dry electrode according to an embodiment of the present invention,

FIG. 4 is a flow diagram of a method for producing an apparatus for acquiring electrophysiological signals according to an embodiment of the present invention,

FIG. 5 is a schematic view of a layer structure of a dry electrode as well as a portion of a line according to an embodiment of the present invention,

FIG. 6 is a schematic view of an inside of the apparatus for acquiring electrophysiological signals according to an embodiment of the present invention,

FIG. 7 is a schematic detailed view of an outside part of the terminal with the connecting elements according to an embodiment of the present invention and

FIG. 8 is a schematic detailed view of an inside part of the terminal realizing the connections between the insulated lines and the connecting elements exposed at the outside of the textile carrier.

### DETAILED DESCRIPTION OF THE INVENTION

In the subsequent description of the embodiments of the present invention, same or equal elements are provided with the same reference numbers in the figures such that their description is inter-exchangeable.

### Apparatus (Strap System) For Acquiring Electrophysiological Signals

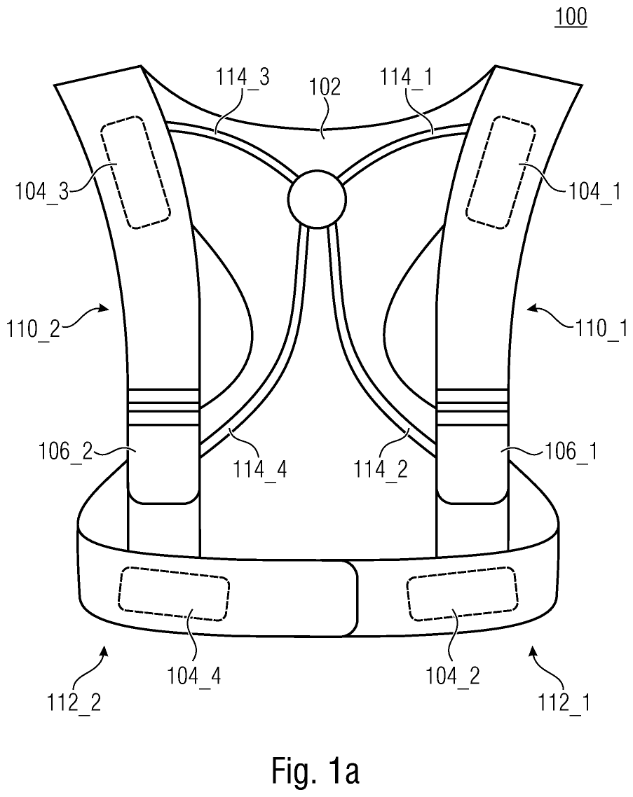

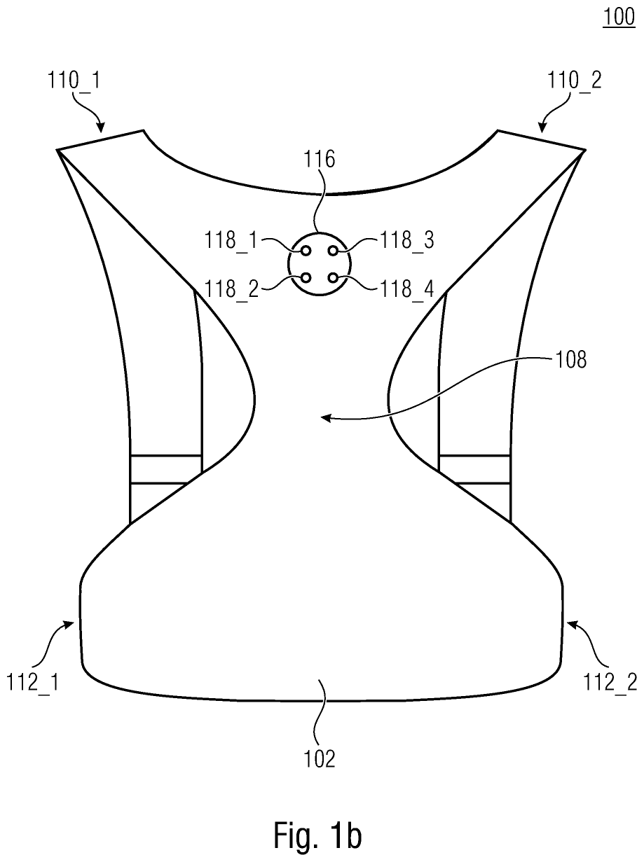

FIG. 1a shows a schematic front view of an apparatus 100 for acquiring electrophysiological signals of a living being (e.g., animal or human being) and FIG. 1b shows a schematic rear view of the apparatus 100 for acquiring electrophysiological signals of a living being according to an embodiment of the present invention.

The apparatus 100 includes a textile carrier 102, at least two dry electrodes 104_1-104_4 attached to the inside of the textile carrier 102 and at least two straps 106_1-106_2 attached to the textile carrier 102. Here, the at least two straps 106_1-106_2 allow adjusting the wearable apparatus 100 to a body of the living being and/or contact pressure of the at least two dry electrodes 104 to a skin of the living being, which can improve, e.g., the quality of the physiological signal detectable by the at least two dry electrodes 104_1-104_4.

In FIGS. 1a and 1b, it is exemplarily assumed that the apparatus 100 comprises four dry electrodes. However, it should be noted that the invention is not limited to such embodiments. Rather, the apparatus 100 can generally comprise n dry electrodes 104_1-104_4, wherein n is a natural number greater than 2, n≥2, such as 2, 3, 4, 5, 6 or more dry electrodes.

As shown exemplarily in FIGS. 1a and 1b, the textile carrier 102 can be made of one (e.g., single) piece of material and can be formed such that the same comprises five areas: central area 108, two upper areas 110_1-110_2, each extending away from the central area 108, and two lateral areas 112_1-112_2, each extending away from the central area 108, wherein, when the apparatus 100 is worn by a living being, the central area 108 of the textile carrier 102 extends across a back area of the living being, the two upper areas 110_1-110_2 of the textile carrier 102 extend, starting from the back area, across respective shoulder areas up to the upper chest areas of the living being and the two lateral areas 112_1-112_2 of the textile carrier 102 extend, starting from the back area, across respective axillary lines up to the respective upper stomach areas or lower chest areas.

The two upper areas 110_1-110_2 of the textile carrier 102 can be connected to the two lateral areas 112_1-112_2 of the textile carrier via two of the at least two straps. For example, a first upper area 110_1 of the textile carrier 102 can be connected to a first lower area 112_1 of the textile carrier 102 via a first strap 106_1, while a second upper area 110_2 of the textile carrier 102 can be connected to a second lower area 112_2 of the textile carrier 102 via a second strap 106_2.

The two lateral areas 112_1-112_2 can be connected via a third strap or alternatively via a connecting element such as Velcro fastener, buckle, clamping strap or push button.

In embodiments, the at least two dry electrodes can be attached to at least two areas of the two upper areas 110_1-110_2 and the two lateral areas 112_1-112_2 of the textile carrier 102 at an inside of the textile carrier 102. For example, as shown in FIGS. 1a and 1b, a first dry electrode 104_1 can be attached to a first upper area 110_1 of the textile carrier 102, a second dry electrode 104_2 to a first lower area 112_1 of the textile carrier 102, the third dry electrode 104_3 to a second upper area of the textile carrier 102 and a fourth dry electrode 104_4 to the second lower area 112_2 of the textile carrier 102.

In embodiments, the at least two dry electrodes 104_1-104_4 can be connected to a terminal 116 attached to an outside of the textile carrier 102 via insulated lines 114_1-114_4, e.g., for providing the electrophysiological signals acquired by the at least two dry electrodes. For this, the terminal 116 can comprise connecting elements 118_1-118_4 guided to the outside, which are connected to the at least two insulated lines.

As shown in FIGS. 1a and 1b, together with the at least two straps 106_1-106_2, the textile carrier 102 can form, for example, the shape of a strap system. When using the strap system for acquiring physiological signals of a human being or a human-like animal (e.g., monkey), the strap system can, for example, have the shape of a vest.

In embodiments, the apparatus shown in FIGS. 1a and 1b can, for example, be used for acquiring an electrocardiogram (ECG) or for monitoring patients with cardiovascular risk constellation or for monitoring patients in cardiological rehabilitation or for telemedical cardio monitoring.

In other words, FIG. 1a shows a front view of the apparatus 100 (strap system) and FIG. 1b a rear view of the apparatus 100. The textile carrier 102 (e.g., in the form of a vest) consists of a single elastic piece of material to which the straps 106_1-106_2 are sewn. The straps 106_1-106_2 are elastic and have a Velcro connection such that when putting the same on, width and even contact pressure of the electrodes 104_1-104_4 can be adapted.

The electrodes 104_1-104_4 are located at the inside of the strap system. For better adherence of the electrodes 104_1-104_4 to the main part, the electrodes have the non-slip surfaces. Contacts 118_1-118_4 for connecting a measurement device are located at the outside of the strap system (see FIG. 1b).

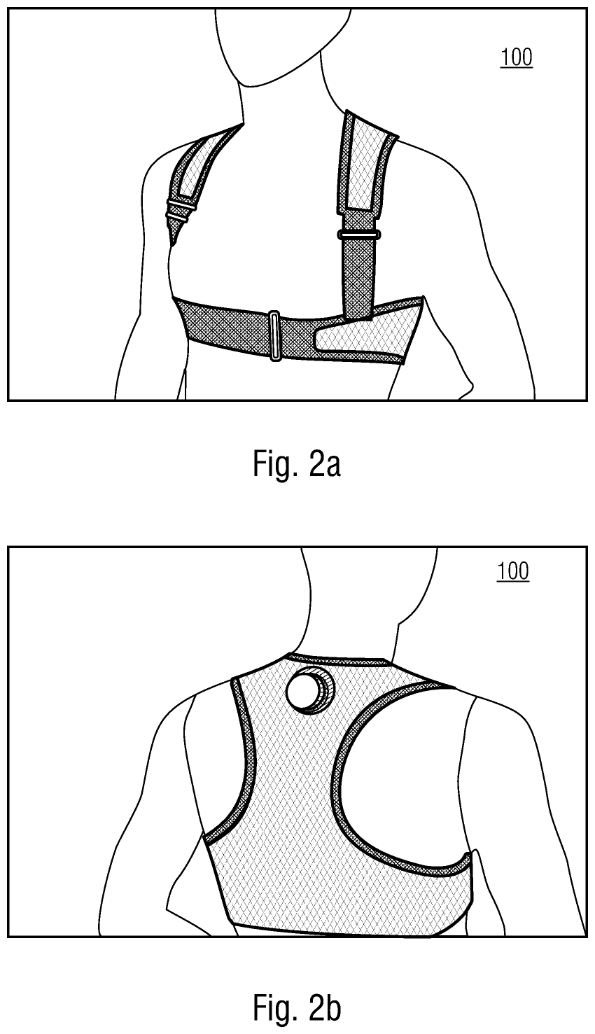

FIG. 2a shows an illustration of a front view of the apparatus 100 when the same is worn exemplarily by a doll as a representative for a living being, while FIG. 2b shows an illustration of a rear view of the apparatus 100 when the same is worn exemplarily by a doll as a representative for a living being.

### Dry Electrodes and Combination of Electrode Materials

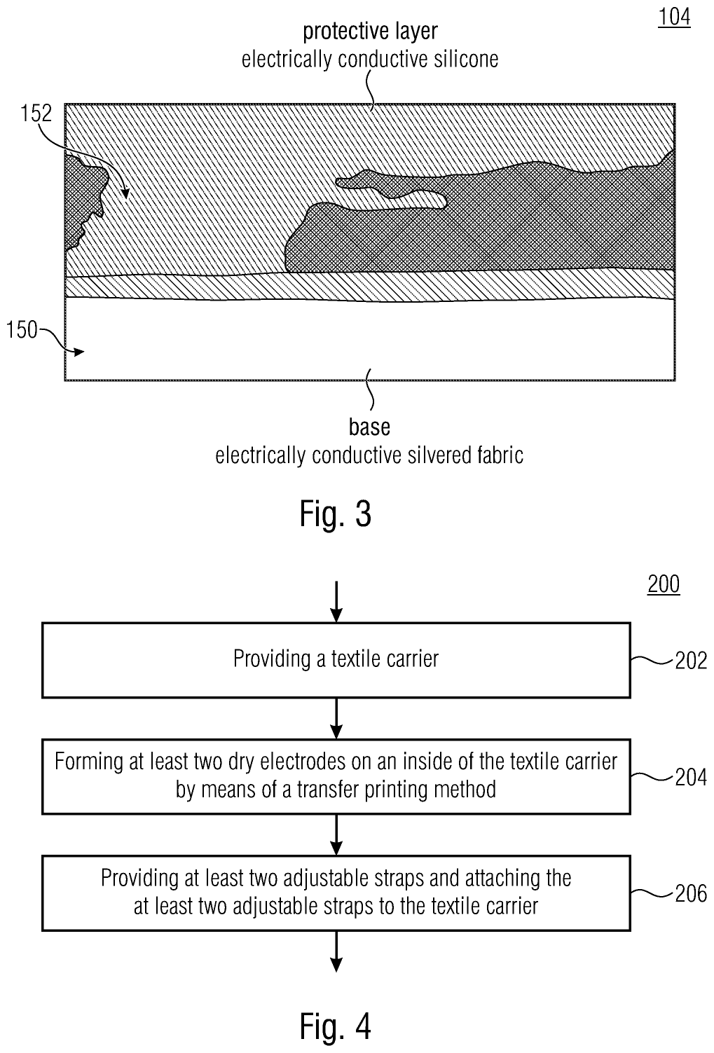

FIG. 3 shows a schematic view of a dry electrode 104 according to an embodiment of the present invention. As can be seen in FIG. 3, the dry electrode 104 comprises a layer 150 of electrically conductive fabric and a layer 152 of electrically conductive polymer covering the layer 150 of conductive fabric.

The requirements for the dry electrodes 104 during the ECG measurement are that the resistance of the dry electrodes 104 is low and remains almost constant over time. Additionally, the dry electrodes should have good adhesion on the skin of the patient. For fulfilling these requirements, a combination of materials can be used in embodiments, as will be discussed below.

In embodiments, as electrically conductive fabric, a silvered knitted fabric can be used as base material. The advantage of this fabric is its improved (e.g., high) electrical conductivity, but silver particles are washed out in open areas of the electrodes during washing. This results in an increase of the electrical resistance, which results in a deterioration of the ECG signal. For preventing washing out of silver particles, in embodiments, this fabric is protected by an electrically conductive polymer, such as a silicone coating (see FIG. 3). Electrically conductive silicone has a lower conductivity than the silvered knitted fabric, but when the same is deposited in a thin layer (e.g., 1 mm or less) on the surface of the silvered knitted fabric, the improved (e.g., high) conductivity of the dry electrode 104 is maintained and simultaneously the dry electrode 104 is protected from washing out of silver particles. Above that, due to its good elasticity, silicone shows good adhesive characteristics on the skin of a living being (e.g., human being or animal). This material combination allows repeated usage of dry electrodes and usage of the same in washable clothes.

### Integration of Materials

FIG. 4 shows a flow diagram of a method 200 for producing apparatus 100 for acquiring electrophysiological signals according to an embodiment of the present invention. The method 200 includes a step 202 of providing a textile carrier. Further, the method 200 includes a step 204 of forming at least two dry electrodes in an inside of the textile carrier by means of a transfer printing method. Further, the method 200 includes a step 206 of providing at least two adjustable straps and attaching the at least two adjustable straps to the textile carrier.

In embodiments, in the step 204 of forming the at least two dry electrodes, the insulated lines connected to the at least two dry electrodes and (optionally) also the terminal connected to the insulated lines can also be formed.

In the following embodiments of step 204 will be described in more detail.

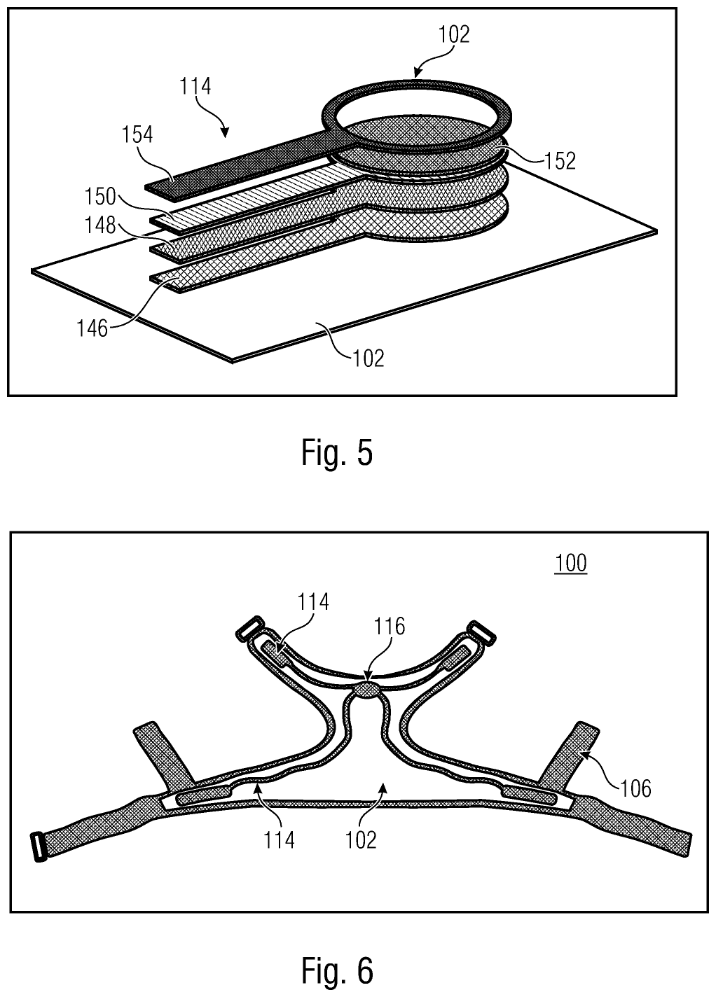

In order to make the apparatus 100 suitable for everyday use and persistent against reusable washing, in embodiments, the transfer printing method can be used. Normally, the transfer printing method is used for decorating and sealing clothes. In embodiments, this method is used to weld all components (dry electrodes, lines, terminals) with the basic textiles (textile carrier) and to seal electrically conductive materials. For that purpose, a multi-layered material structure can be used as will be discussed below based on FIG. 5.

In detail, FIG. 5 shows a schematic view of a layer structure (e.g., multi-layered integration structure) of a dry electrode 104 as well as a portion of a line 114 according to an embodiment of the present invention.

In a preparation phase, all layers for the dry electrode 104 and the insulated line 114 can be provided, e.g., for example cut out according to a template and then positioned on upper of one another. As can be seen in FIG. 5, the layers can be attached in the following order (starting from the textile carrier 102);
Textile carrier 102,
Transfer printing film 146,
Thermoplastic polyurethane film (TPU) 148,
Conductive fabric 150,
Conductive polymer layer (e.g., silicone layer) 152 (only on the conductive fabric 150 in the area of the dry electrode 102) and
Transfer printing film 154 (opened in the area of the dry electrode 102, such that the conductive polymer layer 152 is partly exposed (e.g., only covered by the transfer printing film 154 on the edge)).

After the above layers have been placed, this layer structure (sandwich structure) can be heated under pressure (e.g., 170° C.). The bonding result is illustrated in FIG. 6-8.

Here, FIG. 6 shows a schematic view of an inside of the apparatus 100 according to an embodiment of the present invention. As can be seen in FIG. 6, the dry electrodes 104, insulated lines 114 and an inside part of the terminal 116 are attached on an inside of the textile carrier 102. The inside part of the terminal 116 can realize connections between the insulated lines 114 and the connecting elements that are exposed on the outside of the textile carrier. Further, in FIG. 6, the straps 106 attached to the textile carrier can be seen.

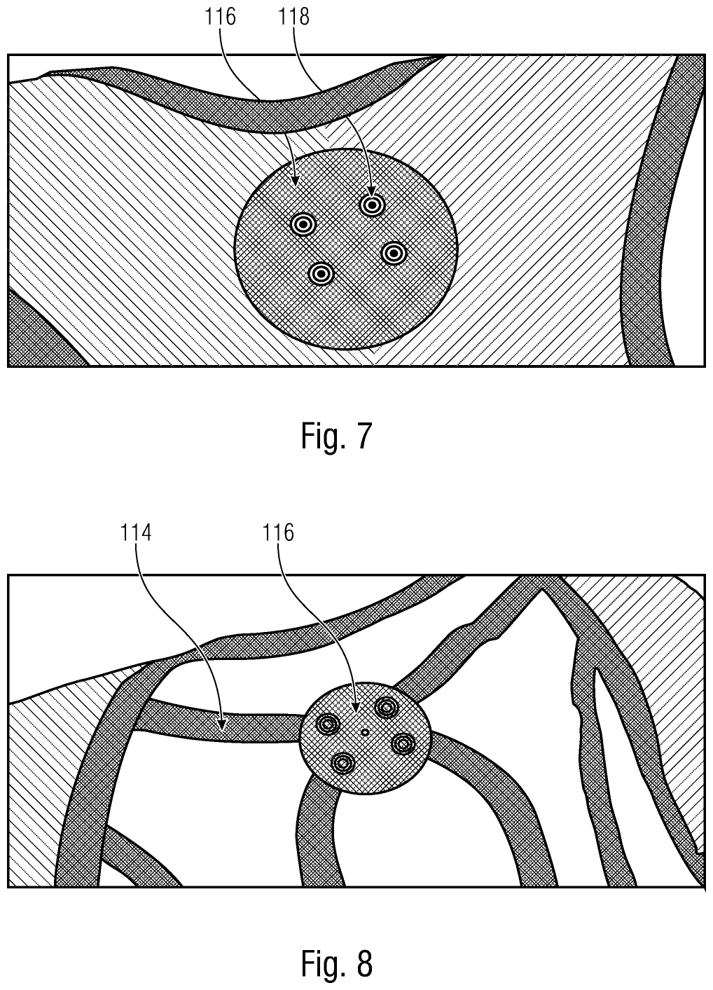

FIG. 7 shows a schematic detailed view of an outside part of the terminal 116 with the connecting elements 118 according to an embodiment of the present invention. The connecting elements can be realized, e.g., by means of push buttons or banana jacks.

FIG. 8 shows a schematic detailed view of an inside part of the terminal 116 realizing the connections between the insulated lines 114 and the connecting elements exposed on the outside of the textile carrier.

The above-described structure of lines is watertight and protects from humidity or sweat during usage.

### Further Embodiments

For capturing physiological signals in a quality that is as high as possible as well as for preventing interfering influences during data acquisition (in particular in application scenarios in mobile everyday life), sufficiently good and reliable adaptation of the primary measurement value sensors (here ECG electrodes) to the human body presents a great challenge. First, embodiments realize a combination of improved (e.g., best possible) material components for a signal acquisition and signaled transmission. Second, embodiments implement requirements for interference stability by the selected type of integration of these materials into a textile carrier. Third, embodiments address and alleviate further critical points by the selected concept (as adjustable strap system), such that sufficiently good signal quality can be provided for different sexes, body sizes and body proportions at all times.

Although some aspects have been described in the context of an apparatus, it is obvious that these aspects also represent a description of the corresponding method, such that a block or device of an apparatus also corresponds to a respective method step or a feature of a method step. Analogously, aspects described in the context of a method step also represent a description of a corresponding block or detail or feature of a corresponding apparatus. Some or all of the method steps may be performed by a hardware apparatus (or using a hardware apparatus), such as a microprocessor, a programmable computer or an electronic circuit. In some embodiments, some or several of the most important method steps may be performed by such an apparatus.

While this invention has been described in terms of several advantageous embodiments, there are alterations, permutations, and equivalents, which fall within the scope of this invention. It should also be noted that there are many alternative ways of implementing the methods and compositions of the present invention. It is therefore intended that the following appended claims be interpreted as including all such alterations, permutations, and equivalents as fall within the true spirit and scope of the present invention.

1. Wearable apparatus for acquiring electrophysiological signals of a living being, comprising:
a textile carrier,
at least two dry electrodes attached to an inside of the textile carrier and
at least two adjustable straps attached to the textile carrier, which allow adjusting the wearable apparatus to the body of the living being and a contact pressure of the at least two dry electrodes on a skin of the living being.

2. Wearable apparatus according to claim 1,
wherein the at least two dry electrodes are at least four dry electrodes for acquiring a multi-channel electrocardiogram of the living being.

3. Wearable apparatus according to claim 1,
wherein the textile carrier is made of one piece of material.

4. Wearable apparatus according to claim 1,
wherein the textile carrier forms the shape of a strap system together with the at least two straps.

5. Wearable apparatus according to claim 1,
wherein the textile carrier comprises a central area, two upper areas, each extending away from the central area and two lateral areas, each extending away from the central area.

6. Wearable apparatus according to claim 5,
wherein, when the apparatus is worn by the living being, the central area of the textile carrier extends across a back area of the living being, the two upper areas of the textile carrier extend, starting from the back area, across respective shoulder areas up to the upper chest areas of the living being and the two lateral areas of the textile carrier extend, starting from the back area, across respective axillary lines up to respective upper stomach areas or lower chest areas.

7. Wearable apparatus according to claim 5,
wherein the two upper areas of the textile carrier can be connected to the two lateral areas of the textile carrier via two of the at least two straps.

8. Wearable apparatus according to claim 5,
wherein the two lateral areas of the textile carrier can be connected to each other via a connecting element,
or wherein the two lateral areas of the textile carrier can be connected to each other via a third strap of the at least two straps.

9. Wearable apparatus according to claim 5,
wherein the at least two dry electrodes are attached to the textile carrier on at least two areas of the two upper areas and the two lateral areas of the textile carrier.

10. Wearable apparatus according to claim 5,
wherein the at least two dry electrodes are at least four dry electrodes,
wherein two dry electrodes of the at least four dry electrodes are attached to an inside of the two upper areas of the textile carrier,
wherein two other dry electrodes of the at least four dry electrodes are attached to an inside of the two lateral areas of the textile carrier.

11. Wearable apparatus according to claim 5,
wherein, when the apparatus is worn by the living being, the two dry electrodes contact clavicle areas [e.g., left and right clavicle areas] or upper areas above a chest area of the living being,
and/or wherein, when the apparatus is worn by the living being, the two other dry electrodes contact abdominal areas [e.g., within an abdominal quadrant] or lower regions below a chest area of the living being.

12. Wearable apparatus according to claim 1,
wherein the textile carrier and/or the at least two straps are elastic.

13. Wearable apparatus according to claim 1,
wherein the at least two dry electrodes each comprise a layer of conductive fabric and a layer of electrically conductive polymer covering the layer of conductive fabric.

14. Wearable apparatus according claim 13,
wherein the layer of electrically conductive polymers is thinner than 1 mm.

15. Wearable apparatus according to claim 13,
wherein layers of the at least two dry electrodes are formed by means of a combination of a screen printing method and a transfer printing method.

16. Wearable apparatus according to claim 1,
wherein the at least two dry electrodes are connected, via insulated lines, to a terminal attached to an outside of the textile carrier.

17. Wearable apparatus according to claim 16,
wherein the insulated lines each comprise a layer of conductive fabric and at least one layer of insulating material covering the layer of conductive fabric.

18. Wearable apparatus according to claim 17,
wherein layers of the insulated lines are formed by means of combination of a screen printing method and a transfer printing method.

19. Wearable apparatus according to claim 16,
wherein the insulated lines are each connected to a connecting element of the terminal guided to the outside.

20. Wearable apparatus according to claim 19,
wherein the terminal comprises at least one layer of insulating material,
wherein a layer of the at least one layer of insulating material is opened in areas of the connecting elements, such that the connecting elements are exposed.

21. Dry electrode for acquiring electrophysiological signals of a living being, comprising:
a layer of conductive fabric and
a layer of electrically conductive polymer covering the layer of conductive fabric.

22. Dry electrode according to claim 21,
wherein the conductive fabric is a silvered fabric.

23. Dry electrode according to claim 21,
wherein the layer of electrically conductive polymer comprises a thickness of less than 1 mm.

24. Dry electrode according to claim 21,
wherein the dry electrode further comprises a thermoplastic polyurethane film,
wherein the layer of conductive fabric is arranged on the thermoplastic polyurethane film.

25. Dry electrode according to claim 21,
wherein the dry electrode is embedded in a transfer printing film layer system of at least two transfer printing films,
wherein the transfer printing film layer system is partly opened in an area adjacent to the layer of electrically conductive polymer, such that the layer of electrically conductive polymer is partly exposed.

26. Dry electrode according to claim 21,
wherein the dry electrode is attached to a textile carrier by means of a transfer printing method.

27. Method for producing a wearable apparatus for acquiring electrophysiological signals of a living being, the method comprising:
providing a textile carrier,
forming at least two dry electrodes on an inside of the textile carrier by means of a transfer printing method,
providing at least two adjustable straps and attaching the at least two adjustable straps to the textile carrier.

28. Method according to claim 27,
wherein forming the at least two dry electrodes comprises:
providing a layer of electrically conductive fabric and
providing a layer of electrically conductive polymer on the layer of electrically conductive fabric, such that the layer of electrically conductive polymer at least partly covers the layer of electrically conductive fabric.

29. Method according to claim 28,
wherein forming the at least two dry electrodes further comprises:
providing a thermoplastic polyurethane film,
wherein the layer of conductive fabric is arranged on the thermoplastic polyurethane film.

30. Method according to claim 28,
wherein forming the at least two dry electrodes further comprises:
providing a first transfer printing film and a second transfer printing film,
wherein the layer of electrically conductive polymer and the layer of electrically conductive fabric are arranged between the first transfer printing film and the second transfer printing film,
wherein the first transfer printing film is arranged on the layer of electrically conductive polymer, wherein the first transfer printing film is partly opened in an area adjacent to the layer of electrically conductive polymer, such that the layer of electrically conductive polymer is partly exposed.

31. Method according to claim 30,
wherein the layer of electrically conductive polymer and layer of electrically conductive fabric are embedded between the first transfer printing film and the second transfer printing film by means of the transfer printing method.

32. Method according to claim 27,
wherein, when forming the at least two dry electrodes, further, at least two insulated lines are formed.

33. Method according to claim 32,
wherein the method further comprises:
forming a terminal on an outside of the textile carrier, wherein the terminal is connected to the at least two insulated lines.

34. Method according to claim 33,
wherein, when forming the terminal, connecting elements guided to the outside are formed, which are each connected to one of the at least two insulated lines.

35. Method according to claim 34,
wherein, when forming the terminal, a further transfer printing film is provided, wherein the further transfer printing film is opened in areas of the connecting elements guided to the outside, such that the connecting elements are exposed, wherein the further transfer printing film is attached to the outside of the textile carrier by means of the transfer printing method to form the terminal.

36. Method for acquiring a multi-channel electrocardiogram of a living being by means of a wearable apparatus for acquiring electrophysiological signals of a living being, the apparatus comprising: a textile carrier, at least two dry electrodes attached to an inside of the textile carrier and at least two adjustable straps attached to the textile carrier, which allow adjusting the wearable apparatus to the body of the living being and a contact pressure of the at least two dry electrodes on a skin of the living being, wherein the at least two dry electrodes are at least four dry electrodes for acquiring a multi-channel electrocardiogram of the living being, the method comprising:
applying a reference signal to the living being with a dry electrode of the at least four dry electrodes of the wearable apparatus,
acquiring at least three electrophysiological signals from the living being using at least three other dry electrodes of the at least four dry electrodes of the wearable apparatus,
processing the acquired at least three electrophysiological signals to attain the multi-channel electrocardiogram of the living being.
